(19) United States
(12) Patent Application Publication
(10) Pub. No.: US 2026/0263569 A1
(43) Pub. Date: Sep. 10, 2026

(54) Targeted Nanomedicine for Treating Arterial Disease

(71) Applicant: The University of Chicago, Chicago, IL (US)

(72) Inventors: Yun Fang, Chicago, IL (US); Siyang Wang, Chicago, IL (US); Matthew Tirrell, Chicago, IL (US); Zhengjie Zhou, Chicago, IL (US)

(21) Appl. No.: 18/875,535

(22) PCT Filed: Jun. 16, 2023
(86) PCT No.: PCT/US2023/068598

§ 371 Date: Dec. 16, 2024

Related U.S. Application Data
(60) Provisional application No. 63/353,468, filed on Jun. 17, 2022.

(51) Int. Cl. A61K 38/46; A61K 9/1271; A61K 47/24; A61K 47/28; A61K 47/34; A61K 47/64; A61K 47/69; A61K 48/00

(52) U.S. Cl. CPC A61K 38/465; A61K 9/1271; A61K 47/24; A61K 47/28; A61K 47/34; A61K 47/64; A61K 47/6911; A61K 48/0041; A61K 48/005; C12Y 301/03004

(57) ABSTRACT

This disclosure relates to lipid nanoparticle composition comprising polyamidoamine (PAMAM) dendrimer (G0-C14), cholesterol, polyethylene glycol 2000 (PEG), 1,2-Distearoyl-sn-glycero-3-phosphoethanolamine-Poly(ethylene glycol) (DSPE-PEG), dioleoylphospha-tidylethanolamine (DOPE), and PLPP3 mRNA encapsulated by the lipid nanoparticle, and methods for treating arterial disease, including, for example, atherosclerosis, peripheral artery disease, ischemic stroke, and stenosis.

### CROSS REFERENCE TO RELATED APPLICATIONS

This application claims the benefit of U.S. Provisional Application No. 63/353,468, filed Jun. 17, 2022, which is incorporated by reference herein in its entirety.

### STATEMENT REGARDING FEDERALLY SPONSORED RESEARCH

This invention was made with government support under grant numbers R01 HL138223, R01 HL136765, and R35HL161244-01 funded by the National Institutes of Health (NIH). The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Jun. 16, 2023, is named “22-0857-WO_SequenceListing.xml” and is 7,877 bytes in size.

### BACKGROUND OF THE DISCLOSURE

### Field of Invention

This disclosure relates to compositions and methods for treating arterial disease, including, for example, atherosclerosis, peripheral artery disease, ischemic stroke, and stenosis.

### Technical Background

Vascular diseases such as atherosclerosis and ischemic stroke are the leading causes of deaths and morbidity in the United States and around the world. Current vascular therapies largely treat systemic risk factors (e.g. hypercholesterolemia and hypertension) and remain suboptimal. One unique feature of vascular diseases is that the vascular complications typically occur in very specific vascular regions of inflammation. For instance, at sites of atypical geometry (arches, branches, and bifurcations), disturbed blood activates endothelial cells to induce local vascular inflammation and cause vascular diseases as demonstrated in Hahn et al. (Nat. Rev. Mol. Cell Biol., 2009, 10 (1), 53-62). Endothelial activation and phenotypic switch of smooth muscle cells (SMC) from a contractile to a synthetic phenotype are major cellular events driving vascular diseases.

Treatments that specifically target diseased blood vessels, such as those of local inflammation and exposed to disturbed blood flow, although remain to be developed, and could revolutionize future cardiovascular therapies.

Targeting molecular mechanisms to treat vascular inflammation in a cell-specific manner will have tremendous potential to treat arterial vascular disease. The present disclosure provides novel lipid nanoparticles and polyelectrolyte complex micelles for gene delivery to target inflamed and/or diseased vasculature. There is a need for a convenient therapeutic avenue to treat arterial vacular disease. Therefore, targeted nanomedicine approaches with tremendous potential to target inflamed endothelial cells and treat arterial disease were developed as described herein.

### SUMMARY OF THE DISCLOSURE

This disclosure describes compositions and methods for treating arterial disease, including, for example, atherosclerosis, peripheral artery disease, ischemic stroke, and stenosis.

In a first aspect, the present disclosure provides a lipid nanoparticle, comprising a VCAM-1 targeting molecule; and an RNA molecule encoding Phospholipid Phosphatase 3 (PLPP3).

In some embodiments of the first aspect, the lipid nanoparticle comprises a polyamidoamine (PAMAM) dendrimer (G0-C₁₄), cholesterol, polyethylene glycol 2000 (PEG), 1,2-Distearoyl-sn-glycero-3-phosphoethanolamine-Poly(ethylene glycol) (DSPE-PEG), dioleoylphospha-tidylethanolamine (DOPE), and PLPP3 mRNA encapsulated by the lipid nanoparticle. In some embodiments, the PEG domain comprises PEG having an average molecular weight of about 1,000 to about 100,000 Daltons. In some embodiments, the VCAM-1 targeting molecule comprises a peptide comprising the amino acid sequence VHPKQHR (SEQ ID NO: 1) or DITWDQLWDLMK (SEQ ID NO: 3). In some embodiments, the VCAM-1 targeted lipid nanoparticle comprises DSPE-PEG-VHPKQHR (SEQ ID NO:6) or DSPE-PEG-DITWDQLWDLMK (SEQ ID NO:7). In some embodiments, the RNA molecule encodes PLPP3 mRNA. In some embodiments, the PLPP3 mRNA is a m1Y-substituted PLPP3 mRNA. In some embodiments, the PLPP3 mRNA comprises a concentration of about 0.01 μM to about 100 μM. In some embodiments, the PLPP3 mRNA comprises a concentration of about 2 μM. In some embodiments, the lipid nanoparticle comprises a molar ratio of the polyamidoamine (PAMAM) dendrimer (G0-C14) to DSPE-PEG is about 2:1 to about 15:1; cholesterol to DSPE-PEG is about 15:1 to about 40:1; and DOPE to DSPE-PEG is about 15:1 to about 30:1. In some embodiments, the lipid nanoparticle comprises molar ratios of PAMAM G0-C14:DOPE:Cholesterol:DSPE-PEG of 5:15:25:1.

In a second aspect, the present disclosure provides a pharmaceutical composition, comprising a therapeutically effective amount of a lipid nanoparticle comprising a VCAM-1 targeting molecule and an RNA molecule encoding PLPP3, and a pharmaceutically acceptable carrier, solvent, adjuvant, and/or diluent. In some embodiments, the pharmaceutical composition is formulated for oral, intravenous, topical, ocular, buccal, systemic, nasal, injection, transdermal, rectal, or vaginal administration. In some embodiments, the pharmaceutical composition is formulated for inhalation, or intramuscular, intraperitoneal, subcutaneous, intravenous administration, or insufflation.

In a third aspect, the present disclosure provides a pharmaceutical composition for delivery of RNA, comprising a lipid nanoparticle comprising a VCAM-1 targeting molecule, and an RNA molecule encoding PLPP3, and pharmaceutically acceptable carrier, wherein the composition is formulated such that once administered to the cardiovascular system, it results in the delivery of the RNA molecule to a inflamed endothelial cells of the cardiovascular system.

In a fourth aspect, the disclosure provides a method of treating an arterial disease in a subject, comprising administering to the subject a therapeutically effective amount of a pharmaceutical composition comprising a lipid nanoparticle comprising a VCAM-1 targeting molecule and an RNA molecule encoding PLPP3, wherein the targeted lipid nanoparticle is preferentially targeted to inflamed endothelial cells associated with the arterial disease and reducing inflammation at the site of the inflamed endothelial cells. In some embodiments of the fourth aspect, the arterial disease comprises one or more of atherosclerosis, peripheral artery disease, ischemic stroke, and stenosis. In some embodiments of the fourth aspect, the method results in an enhancement of PLPP3 mRNA at the site of the inflamed endothelial cells compared to a control. In some embodiments, the method results in one or more of an increase of at least about 2 fold to at least about 1000 fold of the RNA encoding PLPP3 compared to a control and/or an increase of at least about 2 fold to at least about 200 fold of PLPP3 protein compared to a control at the site of the inflamed endothelial cells. In certain embodiments, the method further includes stimulating endothelial growth at the site of the wound. In some embodiments, the probability of survival of the individual is at least about 10% greater than an expected probability of survival without administration of the pharmaceutical composition.

In certain embodiments, the lipid nanoparticle compositions disclosed herein can be administered to a subject having arterial disease. In some embodiments, the lipid nanoparticle compositions disclosed herein reduce artherosclerosis in the patient.

In a fifth aspect, the disclosure provides a polyelectrolyte complex micelle (PCM), comprising a PDGFRB targeting molecule and an RNA molecule. In some embodiments, the PCM comprises a polyethylene glycol (PEG) domain, and poly-lysine, wherein the RNA molecule is a microRNA. In some embodiments of the PCM, the PEG domain comprises PEG having an average molecular weight of about 1,000 to about 100,000 Daltons. In some embodiments of the PCM, the PDGFRB targeting molecule comprises a peptide comprising the amino acid sequence CSRNLIDC (SEQ ID NO: 4), a peptide described by Beljaars et al. (Biochem. Pharmacol. 2003, 66 (7), 1307-1317). In some embodiments of the PCM, the polyelectrolyte complex micelle comprises CSRNLIDC (SEQ ID NO: 4), the PEG domain, and poly-lysine, wherein the PCM encapsulates the RNA molecule. In some embodiments, the RNA molecule is a microRNA encoding a microRNA145 mimic. In some embodiments, the PCM encapsulates about 0.01 μM to about 100 μM of the RNA molecule.

In a sixth aspect, the disclosure provides, a pharmaceutical composition comprising a therapeutically effective amount of a polyelectrolyte complex micelle comprising a PDGFRB targeting molecule, a PEG domain, and poly-lysine, wherein the polyelectrolyte complex micelle encapsulates the RNA molecule; and a pharmaceutically acceptable carrier, solvent, adjuvant, and/or diluent. In some embodiments, the pharmaceutical composition is formulated for inhalation, insufflation, oral, intravenous, topical, ocular, buccal, systemic, nasal, injection, transdermal, rectal, or vaginal administration. In some embodiments, the pharmaceutical composition is formulated for inhalation or intravenous administration. In some embodiments, the pharmaceutical composition is for cardiovascular delivery of an RNA molecule comprising a polyelectrolyte complex micelle comprising a PDGFRB targeting molecule comprising the amino acid sequence CSRNLIDC (SEQ ID NO: 4); a PEG domain; and poly-lysine; wherein the RNA molecule is a microRNA encoding a microRNA145 mimic; and a pharmaceutically acceptable carrier, wherein the composition is formulated such that once administered to the cardiovascular system, it results in the delivery of the RNA molecule to a subject's cardiovascular system.

In a seventh aspect, the disclosure provides, a method of treating an arterial disease in a subject, comprising administering to the subject a therapeutically effective amount of the pharmaceutical composition as disclosed herein, wherein the polyelectrolyte complex micelle is preferentially targeted to smooth muscle cells (SMC) associated with the arterial vascular disease; and reducing SMC proliferation and migration, decreasing synthetic SMC phenotype, and increasing the contractile phenotype of SMC at the site of diseased blood vessels. In some embodiments of the method, the arterial disease comprises one or more of atherosclerosis, peripheral artery disease, ischemic stroke, and stenosis. In some embodiments of the method, the method results in one or more of an increase of at least about 2 fold to at least about 1000 fold of the miRNA145 in SMC compared to a control at the site of vascular disease. In some embodiments of the method, the probability of survival of the subject is at least about 10% greater than an expected probability of survival without administration of the pharmaceutical composition.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1 shows a novel VCAM1-targeting lipid nanoparticle that was engineered to effectively encapsulate and deliver messenger RNA (mRNA) of PLPP3 to activated endothelial cells. VCAM1-targeting lipid nanoparticles were characterized by Transmission electron microscopy (TEM) and dynamic light scattering (DLS).

FIG. 2 shows atherosclerotic disease is the leading cause of morbidity and mortality worldwide. Atherosclerosis, thickening and hardening of the vascular walls, develops preferentially at sites of curvature, branching, and bifurcation in blood vessels where endothelial cells are activated by local disturbed flow and smooth muscle cells (SMC) undergo a phenotypic switch from a contractile to a synthetic phenotype. Genome-wide association studies (GWAS) from Schunkert et al. (Nat. Genet., 2011, 43 (4), 333-338) have identified chromosome 1p32.2 (labeled as PLPP3/PPAP2B) as one of the most strongly associated human loci with susceptibility to atherosclerosis.

FIG. 3 shows disturbed blood flow and atherosclerosis risk allele at chromosome 1p32.2 converge to inhibit PLPP3 expression in activated endothelial cells expressing vascular cell adhesion molecule 1 (VCAM-1), leading to atherosclerosis demonstrated by Krause et al. (Proc. Natl. Acad. Sci. U.S.A, 2018, 115 (48), 11349-11358) and Wu et al. (Circ. Res., 2015, 117 (4) 41-53).

FIGS. 4A-4B show that VCAM1-targeting lipid nanoparticles which encapsulate functional PLPP3 mRNA significantly reduce disturbed flow-induced atherosclerosis/stenosis in the ligated left carotid artery in mice. Atherosclerosis/stenosis was induced in the left carotid artery by partial carotid ligation (PCL). Experimental design: Mouse carotid atherosclerosis was induced in the left carotid artery by partial carotid ligation (PCL) in ApoE-deficient mice (Apoe−/−) mice fed with high fat diet (HFD). Functional PLPP3 mRNAs or control non-functional mutant PLPP3 mRNAs encapsulated in the VCAM1-targeting lipid nanoparticles were intravenously injected to the Apoe−/− mice subjected to PCL and HFD. Carotid atherosclerosis in the ligated left carotid artery in Apoe−/− mice was not significantly affected by the three injections of Phosphate-buffered saline (PBS) or VCAM1-targeting lipid nanoparticles encapsulating non-functional mutant PLPP3 mRNAs. In sharp contrast, atherosclerosis was significantly reduced in in Apoe−/− mice subjected to injections of VCAM1-targeting lipid nanoparticle encapsulating functional PLPP3 mRNAs. Three injections of PBS or lipid nanoparticles were conducted (FIG. 4A). PBS: Three injections of PBS; PLPP3-MUT: Three injections of VCAM1-targeting lipid nanoparticles which encapsulate mutant (Mut) non-functional PLPP3 mRNA. PLPP3-WT: Three injections of VCAM1-targeting lipid nanoparticles which encapsulate wild-type (WT) functional PLPP3 mRNA significantly reduce disturbed flow-induced atherosclerosis/stenosis in the ligated left carotid artery in mice (FIG. 4B).

FIG. 5 shows that delivery of functional messenger RNA (mRNA) to activated vascular endothelium by VCAM1-targeting lipid nanoparticles in vivo. Partial carotid artery ligation was conducted in mice to induce acute disturbed flow in the left carotid artery (LCA) which leads to endothelial inflammation. En face images of ligated left carotid arteries in mice subjected to an injection of mScarlet mRNA-encapsulated, VCAM1-targeting lipid nanoparticles. This result demonstrates that the VCAM1-targeting lipid nanoparticles effectively deliver in vivo to inflamed endothelial cells, functional mScarlet mRNA that can be translated to proteins.

FIG. 6 shows that delivery of functional PLPP3 mRNA to activated vascular endothelium by VCAM1-targeting lipid nanoparticles in vivo. Partial carotid artery ligation was conducted in mice to induce acute disturbed flow in the left carotid artery (LCA) which leads to endothelial inflammation. Real-time PCR detected a significant increase of PLPP3 mRNA expression in inflamed endothelial cells in mice subjected to an injection of PLPP3 mRNA-encapsulated, VCAM1-targeting lipid nanoparticles. Specifically, PLPP3 mRNA is significantly increased in the inflamed endothelial cells in the ligated carotid artery where local disturbed blood flow activates endothelial cells.

FIG. 7 shows the formulation of PDGFRB targeting polyelectrolyte complex micelles (PCMs) that encapsulate small RNAs, such as miR-145 mimics.

FIGS. 8A-8B show the characterizations of PDGFRB targeting polyelectrolyte complex micelles (PCM) encapsulating miR-145 mimics. (FIG. 8A) Hydrodynamic radius of miR-145 mimics encapsulated, PDGFRB targeting PCMs distributed between about 40 to 80 nm as demonstrated by the Dynamic light scattering (DLS) and Transmission electron microscopy (TEM). Dynamic light scattering (DLS) was collected using Wyatt Technology's Mobius™ with 532 nm laser at a constant detector angle of 163.5 degrees under simultaneous DLS and PALS mode with 5s acquisition time at 2V amplitude, 10 Hz for 5 repeated measurements at 25° C. The average radius of PCMs is 58.6±4 nm, with percent and the zeta potential of PCMs is 4.7±2 mV. The encapsulation percentage of miR-145 mimics was characterized by RiboGreen free RNA detection assay resulting in a 95.2% encapsulation efficiency. (FIG. 8B) Transmission electron microscopy (TEM) image of PDGFRB targeting PCMs was collected with Ted Pella carbon coated 400 mesh copper grids using FEI Tecnai F30 electron microscope at 300 kV.

FIG. 9 shows that miR-145 mimics encapsulated, PDGFRB targeting polyelectrolyte complex micelles (PCM) effectively achieved targeted delivery of miR-145 mimics to smooth muscle cells (SMC) in vitro.

FIG. 10 shows that PDGFRB targeting polyelectrolyte complex micelles (PCM) effectively deliver miR-145 mimics to smooth muscle cell-enriched arterial media and adventitia in vivo in mouse carotid artery subjected to partial carotid ligation.

### DETAILED DESCRIPTION

Provided herein are compositions and methods for treating arterial disease, including, for example, atherosclerosis, peripheral artery disease, ischemic stroke, and stenosis.

As used herein, the term “arterial disease” refers to vascular disease that affects the arteries of a subject. Arterial diseases can include, but are not limited to: Abdominal Aortic Aneurysm, Thoracic Aortic Aneurysm, Coronary Artery Disease, Carotid Artery Disease, Peripheral Arterial Disease, Vertebrobasilar Disease, Renal Vascular Disease, Thoracic Outlet Syndrome, and Subclavian Steal Syndrome. In some embodiments, arterial disease includes atherosclerosis, peripheral artery disease, ischemic stroke, and stenosis.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indictates otherwise.

In some embodiments, percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

As used herein, the terms “therapeutically effective amount” or “effective amount” refer to that amount of a therapeutic agent, such as a lipid nanoparticle comprising an RNA molecule encoding PLPP3 and/or a polyelectrolyte complex micelle comprising an miRNA 145 mimic, which when administered to a subject, is sufficient to effect treatment (e.g., improve symptoms) for a disease or disorder described herein, such as, for example, arterial disease, vascular disease, including, for example, atherosclerosis, peripheral artery diseaese, ischemic stroke, diseased blood vessels, and stenosis. The amount of a compound which constitutes a “therapeutically effective amount” or “effective amount” can vary depending on the compound, the disorder and its severity, and the age, weight, sex, and genetic background of the subject to be treated, but can be determined by one of ordinary skill in the art.

As used herein, the terms “treating” or “treatment” refer to the treatment of a disease or disorder described herein, in a subject, preferably a human, and includes inhibiting, relieving, ameliorating, or slowing progression of the disease or disorder or one or more symptoms of the disease or disorder.

As used herein, the term “subject” refers to a warm blooded animal such as a mammal, preferably a human, which is afflicted with, or has the potential to be afflicted with one or more diseases and disorders described herein.

As used herein, the term “pharmaceutical composition” refers to a composition that includes one or more therapeutic agents disclosed herein, such as an RNA molecule encoding PLPP3 and/or an miRNA145 mimic, a pharmaceutically acceptable carrier, a solvent, an adjuvant, and/or a diluent, or any combination thereof.

In view of the present disclosure, the methods and compositions described herein can be configured by the person of ordinary skill in the art to meet the desired need. In general, the disclosed materials and methods provide improvements in treating arterial disease as described herein.

Disclosed herein is a targeted approach to restore PLPP3 mRNA in inflamed endothelium to treat aterial disease in a subject and/or to restore miRNA 145 to treat aterial disease in a subject. The engineered targeted lipid nanoparticles successfully deliver PLPP3 mRNA to inflamed endothelium via a targeting peptide against Vascular Cell Adhesion Molecule 1 (VCAM-1). The engineered targeted PCM successfully deliver a miRNA145 mimic to smooth muscle cells via a targeting peptide against PDGFRB. Preliminary data demonstrated that this targeting strategy toward inflamed endothelium reduces artherosclerosis. N1-methylpseudouridine (m1\′) substitution of the uridine, an RNA modification associated with enhanced protein expression of the PLPP3 mRNA.

The significance of the present disclosure includes at least two aspects. First, it provides novel nanomedicine approaches to treat arterial disease with unmet medical need. Second, it integrates targeted nanomedicine and RNA therapeutics to create a new avenue for the treatment of various arterial diseases including atherosclerosis, peripheral artery disease, ischemic stroke, and stenosis. This disclosure further provides, in part, a peptide-targeted polyelectrolyte complex using lipid nanoparticles or polyelectrolyte complex micelles to deliver therapeutic RNA to endothelial cells or smooth muscle cells. In some embodiments, enhancing PLPP3 mRNA expression at the site of the inflamed endothelial cells. In some embodiments, enhancing miRNA145 levels in smooth muscle cells at the site of vascular disease.

### Compositions

In some embodiments, pharmaceutical compositions contemplated herein include a therapeutically effective amount of a targeted lipid nanoparticle including one or more inhibitors of endothelial inflammation, such as, for example, an RNA molecule encoding PLPP3. Such compositions may further include an appropriate pharmaceutically acceptable carrier, solvent, adjuvant, diluent, or any combination thereof. The exact nature of the carrier, solvent, adjuvant, or diluent will depend upon the desired use (e.g., route of administration) for the composition, and may range from being suitable or acceptable for veterinary uses to being suitable or acceptable for human use.

In some embodiments, pharmaceutical compositions contemplated herein include a therapeutically effective amount of a polyelectrolyte complex micelle including an miRNA145 mimic. Such compositions may further include an appropriate pharmaceutically acceptable carrier, solvent, adjuvant, diluent, or any combination thereof. The exact nature of the carrier, solvent, adjuvant, or diluent will depend upon the desired use (e.g., route of administration) for the composition, and may range from being suitable or acceptable for veterinary uses to being suitable or acceptable for human use.

In some embodiments, pharmaceutical compositions contemplated herein include one or more lipid nanoparticles that carry the one or more mRNA enhancers, such as an RNA molecule encoding PLPP3, for example, inside the lipid nanoparticle, attached to an external surface of the lipid nanoparticle, or both. In some embodiments, the lipid nanoparticles include one or more targeting moeities attached thereto to enable targeted delivery of the lipid nanoparticle to a desired location. For example, the targeting moeity can target the lipid nanoparticle to a site of endothelial inflammation associated with arterial disease or artery disorder or arterial wound.

In some embodiments, pharmaceutical compositions contemplated herein include one or more polyelectrolyte complex micelles that carry the one or more mRNA regulators, such as miRNA145 mimics, for example, inside the PCM, attached to an external surface of the PCM, or both. In some embodiments, the PCM include one or more targeting moeities attached thereto to enable targeted delivery of the PCM to a desired location. For example, the targeting moeity can target the PCM to smooth muscle cells at a site of vascular injury associated with arterial disease or artery disorder or arterial wound.

Any therapeutic RNA is contemplated herein. For example, contemplated mRNAs include PLPP3 mRNA. In another example, the therapeutic RNA is a miRNA or an miRNA mimic, for example, an miRNA145 mimic.

Such compositions optionally include secondary therapeutic agents (possibly also carried on or in contemplated lipid nanoparticles or PCMs).

In some embodiments, the RNA molecule encoding PLPP3 mRNA or the miRNA145 mimic, of the present disclosure can be administered through a variety of routes and in various compositions. For example, pharmaceutical compositions containing the RNA molecule encoding PLPP3 mRNA or the miRNA145 mimic, can be formulated for oral, intravenous, topical, ocular, buccal, systemic, nasal, injection, transdermal, rectal, or vaginal administration, or formulated in a form suitable for administration by inhalation or insufflation. In some embodiments of the present disclosure, administration is oral or intravenous.

A variety of dosage schedules is contemplated by the present disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease. In some embodiments of the present disclosure, one or more an RNA molecules encoding PLPP3 mRNA or one or more miRNAs comprising an miRNA145 mimic, are administered orally or intravenously, and the subject is dosed on a daily basis.

The therapeutic agents (also referred to as “compounds” herein) described herein (e.g., polyelectrolyte complex micelles, lipid nanoparticles, VCAM-1 targeting molecule, PDGFRB targeting molecule, mRNA, miRNA), or compositions thereof, will generally be used in an amount effective to achieve the intended result, for example, in an amount effective to provide a therapeutic benefit to subject having the particular disease being treated. As used herein, therapeutic benefit refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease.

Determination of an effective dosage of compound(s) for a particular disease and/or mode of administration is well known. Effective dosages can be estimated initially from in vitro activity and metabolism assays. For example, an initial dosage of compound for use in a subject can be formulated to achieve a circulating blood or serum concentration of the metabolite active compound that is at or above an IC₅₀ of the particular compound as measured in an in vitro assay. Calculating dosages to achieve such circulating blood or serum concentrations taking into account the bioavailability of the particular compound via a given route of administration is well within the capabilities of a skilled artisan. Initial dosages of compound can also be estimated from in vivo data, such as from an appropriate animal model.

Dosage amounts of an RNA molecule encoding PLPP3 mRNA or the miRNA145 mimic can be in the range of from about 0.0001 mg/kg/day, about 0.001 mg/kg/day, or about 0.01 mg/kg/day, or about 0.1 mg/kg/day, or about 1.0 mg/kg/day, or about 10 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, including particular condition being treated, the severity of existing or anticipated physiological dysfunction, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

For example, a dosage contemplated herein can include a single volume of about 0.1, 0.2, 0.3, 0.4, 0.5, 0.6, 0.7, 0.8, 0.9, 1.0, 1.5, 2.0, 2.5, or 3.0 mL of a pharmaceutical composition having a concentration of mRNA encoding PLPP3 or the miRNA comprising an miRNA145 mimic at about 0.00001, 0001, 0.001, 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 10, 15, 20, 50, 100, 200, 500, or 1000 mM in a pharmaceutically acceptable carrier.

### Polyelectrolyte Complex Micelles (PCM)

The present disclosure contemplates use of polyelectrolyte complex micelles to deliver therapeutic agents. Polymers that bear charge in an aqueous environment are called polyelectrolytes. When oppositely charged polymers are mixed under the right conditions, they form complexes. Polyelectrolytes, including at least one attached to a non-charged, water soluble block, can be mixed at a stoichiometric charge ratio with an oppositely charged homopolymer to form particles of a relatively compact core surrounded by a dilute corona of neutral water soluble block. These nanometer-sized particles are called polyelectrolyte complex micelles, polyion complex micelles, interpolyelectrolyte complex micelles, complex coacervate core micelles, or polyelectrolyte micelles. Polyelectrolyte complexes composed of nucleic acids and positively charged polymers have been explored as a possibility to neutralize the charge on the molecule and protect it from enzymatic degradation. Polyelectrolyte complex micelles have great potential as gene delivery vehicles because of their ability to encapsulate charged nucleic acids, forming a core by neutralizing their charge, while simultaneously protecting the nucleic acids from non-specific interactions and enzymatic degradation. Furthermore, to enhance specificity and transfection efficiency, polyelectrolyte complex micelles can be modified to include targeting capabilities.

The contemplated polyelectrolyte micelles can comprise polyethylene glycol (PEG) domains. PEG domains prevent macrophase separation, stabilizing the micelles. The domains further protect the nanoparticles from recognition by the reticuloendothelial system in the body. The PEG domain can be comprised of PEG having an average molecular weight of about 1,000 to about 100,000 Daltons (Da).

Contemplated polyelectrolyte micelles for use herein include, for example, polyelectrolytes that can effectively incorporate negatively-charged nucleotides in the core and functionally display tissue-targeting peptides on the surface.

These self-assembled nano-scale carriers (about 120 nm in diameter) are formed by electrostatic interaction between two oppositely-charged polymers. Polyethylene glycol (PEG)-2000 was conjugated with poly-Lysine (Poly-Lysine with about 50 repeat units) at one end and the targeting peptide at the other end. In some embodiments, the nanoparticles comprise lipid grafted polyamidoamine (PAMAM) dendrimer (G0-C14), cholesterol, 1,2-Distearoyl-sn-glycero-3-phosphoethanolamine-Poly(ethylene glycol) (DSPE-PEG), dioleoylphospha-tidylethanolamine (DOPE), and PLPP3 mRNA and conjugated with targeting peptides to functionalize the nanoparticles to bind specific cell membrane molecules. In some embodiments, the contemplated nanoparticle can comprise a polyelectrolyte complex micelle, a PDGFRB targeting molecule, and an RNA molecule of miRNA145 mimics.

In some embodiments, the contemplated nanoparticle can comprise a polyelectrolyte complex micelle, a VCAM-1 targeting molecule, and an RNA molecule encoding PLPP3. Negatively-charged nucleotides are neutralized by poly-lysine and encapsulated in the cores of the polyelectrolyte complex micelle. This approach offers multiple advantages, including: (i) the nano-scale of micelles significantly increases the surface area:volume ratio that can enhance specific targeting, and (ii) the self-assembling feature of the polyelectrolytes eliminates the use of chemical cross-linking agents, thereby reducing possible toxicities. lower than the therapeutically effective amount of the naked (non-targeted) therapeutic agent.

Lipid nanoparticles (LNP) are colloidal dispersions that are composed of one or more lipid-bilayers that surround an aqueous core. The ability of lipid nanoparticles to encapusulate lipophilic and hydrophlic drugs have allowed these vesicles to be useful drug delivery systsems. Important physicochemical properties of lipid nanoparticles such as the hydrodynamic diameter or particle size, surface charge (typically measured as zeta-potential), lipid-packing, bilayer lamellarity, encapsulation efficiency, drug encapsulation, molecular loading and external modifications (such as polymer coatings and targeting moiety incorporation) are necessary to accurately control and measure to properly manufacture a pharmaceutical drug product. Lipid nanoparticles can be formed to have a hydrodynamic diameter (in nanometers [d.nm]) ranging from approximately 30 d.nm to over 500 d.nm. For lipid nanoparticles that are less than 500 d.nm, these particles exhibit Brownian motion and remain as a colloidal dispersion since the thermal motion of the particles overcome gravitational forces that would otherwise increase the likelihood of sedimentation.

Additional micelles are contemplated for use herein, such as those disclosed in International Application No. PCT/US2006/020760 (U.S. Pat. No. 9,505,867), Vieregg et al. (J. Am. Chem. Soc. 2018, 140, 1632-1638), Lueckheide et al. (Nano Lett. 2018, 18, 7111-7117), and Marras et al. (Polymers 2019, 11, 83), each of which is incorporated by reference in its entirety.

### Targeting Molecules

The present disclosure contemplates use of targeting molecules (or targeting moieties) with the lipid nanoparticles or PCM nanoparticles disclosed herein for targeted delivery of therapeutic compositions, such as an RNA molecule encoding PLPP3 mRNA or miRNA145 mimics, or for incorporation into pharmaceutical compositions as described herein. Targeting molecules can be VCAM1-targeting molecules. Targeting molecules can include peptides such as VHPKQHR (SEQ ID NO: 1), which was identified via phage display and allows for targeting of inflamed endothelial cells through VCAM-1. Peptide targeting molecules further include the amino acid sequence DITWDQLWDLMK (SEQ ID NO: 3), which allows for the targeting E-selectin. E-selectin is a cell adhesion molecule expressed only on cytokine-activated endothelial cells. Targeting molecules can be PDGFRB-targeting molecules. Targeting molecules can include peptides such as CSRNLIDC (SEQ ID NO: 4), which is a cyclic peptide with a disulfide bond between cysteines that binds specifically to PDGFRB.

The expression of vascular cell adhesion molecule 1 (VCAM1) is low in healthy endothelium but increases in inflamed endothelial cells (ECs). To achieve effective targeting to VCAM1-expressing ECs, the nanoparticles are functionalized with a VCAMI binding peptide that has been shown to facilitate VCAM1-mediated intracellular internalization of nano-materials in endothelium in vitro and in vivo. To achieve effective targeting to smooth muscle cell (SMC), the nanoparticles are functionalized with a PDGFRB-targeting peptide that binds to PDGFRB which is highly expressed in smooth muscle cells.

In some embodiments, contemplated targeting peptides are positioned at the periphery of the corona of the nanoparticles.

In one embodiment, a contemplated targeted lipid nanoparticle containing an RNA molecule encoding PLPP3 (e.g., about 2 μM or more) is DSPE-PEG-VHPKQHR (SEQ ID NO: 6) or DSPE-PEG-DITWDQLWDLMK (SEQ ID NO:7).

In some embodiments, contemplated nanoparticles containing an RNA molecule encoding PLPP3 mRNA or miRNA145 mimics exhibit a polydispersity of about 0.1 to about 0.3.

In some embodiments, contemplated lipid nanoparticles containing an RNA molecule encoding PLPP3 or the PCMs containing a miRNA molecule contained with the core exhibit a spherical shape and have a diameter (in nanometers, nm) of about 10, 11, 12, 13, 14, 15, 16, 17, 18, 19, 20, 21, 22, 23, 24, 25, 30, 35, 40, 45, or 50 nm. In some embodiments, contemplated lipid Nanoparticles or PCMs exhibit a spherical shape and have a diameter (in nanometers, nm) of about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 65, about 70, about 75, about 80, about 85, about 90, about 95, about 100, about 105, about 110, about 115, about 120, or about 125 nm. In some embodiments, the nanoparticles have a radius of about 40 nm to about 80 nm.

The nanoparticle delivery system of the present disclosure possesses multiple advantages compared with other non-targeting platforms. The first advantage is higher stability: the therapeutically active components (e.g., an RNA molecule encoding PLPP3, or miRNA145 mimics, or other nucleic acid based therapeutics) can be encapsulated in the inner core of the lipid nanoaprticles or polyelectrolyte complex micelles and can therefore be protected by the outer layer of biocompatible polymers. Through this approach, first, the degradation of nucleotides of the RNA molecule encoding PLPP3 by serum nucleases is prevented; second, the nanoparticles are capable of escaping renal clearance; and third, the immunogenic responses are avoided. The second advantage is higher safety: cell-targeting peptides (e.g., those targeting to Vascular Cell Adhesion Molecule 1 (VCAM-1), or PDGFRB) are covalently conjugated on the periphery of the nanaoparticles, which significantly reduces cytotoxicity and increases circulation time by circumventing nonspecific interaction with serum components. The third advantage is higher specificity: with the defined chemical structures of the targeting peptides, the nanoparticles are able to bind specific receptors and penetrate targeted cells. The final advantage is higher scalability. This approach does not require chemical modifications on nucleotides for conjugation. The synthesis of the core components in these nanoaprticles is highly automated. In addition, the targeting peptides are easily changeable to target different receptors.

Further, as shown herein, the use of targeted nanoparticles permits use of a lower amount of a therapeutic agent for the treatment of arterial disease or arterial wound due to the specific targeting of the therapeutic agent to the site of the arterial disease or arterial wound. In this way, use of targeted nanoparticles can significantly lower the dosage of a therapeutic agent required to treat arterial disease or arterial wound, which can significantly reduce costs associated with the treatment. For example, a therapeutically effective amount of a therapeutic agent to be delivered by a targeted nanoparticle can be at least about 10, 20, 30, 40, or 50%.

### Methods

In some embodiments, methods of treating and/or preventing arterial disease or arterial wound in a subject in need thereof include administering to the subject a therapeutically effective amount of one or more PLPP3 mRNA molecules and/or one or more miRNA145 mimics. Treatable and/or preventable arterial disease, can include, for example, atherosclerosis, peripheral artery disease, ischemic stroke, and stenosis.

In some embodiments, therapeutic methods contemplated herein can also treat and/or prevent complications associated with or promote endothelial wound healing (e.g., caused by arterial disease or arterial wound) by administering to the subject a therapeutically effective amount of one or more PLPP3 mRNA molecules and/or one or more miRNA145 mimics. For example, treatment and/or prevention of complications associated with endothelial wound healing is associated with the reduction of inflammation at the site of the wound and the stimulation of endothelial growth.

In some embodiments, therapeutic methods contemplated herein can also accelerate endothelial growth to treat wound healing (e.g., caused by stroke or atherosclerosis) by administering to the subject a therapeutically effective amount of one or more PLPP3 mRNA molecules and/or one or more miRNA145 mimics.

The present disclosure contemplates a variety of methods of administering the therapeutic agents, targeting molecules, lipid nanoparticles, and/or polyelectrolyte complex micells as disclosed herein, including local, oral, nasal, rectal, intravaginal, topical, subcutaneous, intradermal, intramuscular (IM), intravenous (IV), intrathecal (IT), intracerebral, epidural, or intracranial administration. Local, in situ administration of these compositions is contemplated.

The present disclosure contemplates methods that result in a variety of indications of improvement for arterial disease in the patient.

### EXAMPLES

The Examples that follow are illustrative of specific embodiments of the disclosure, and various uses thereof. They are set forth for explanatory purposes only and should not be construed as limiting the scope of the disclosure in any way.

The present disclosure leverages unique, cell type-specific biology that was discovered to target specific cell types in arterial disease. Arterial disease treatments do not directly target dysfunctional/inflammed endothelium, underscoring an unmet medical need

### Example 1. VCAM1-Targeting Lipid Nanoparticles which Encapsulate Functional PLPP3 mRNA Significantly Reduce Disturbed Flow-Induced Atherosclerosis/Stenosis in the Ligated Left Carotid Artery in Mice

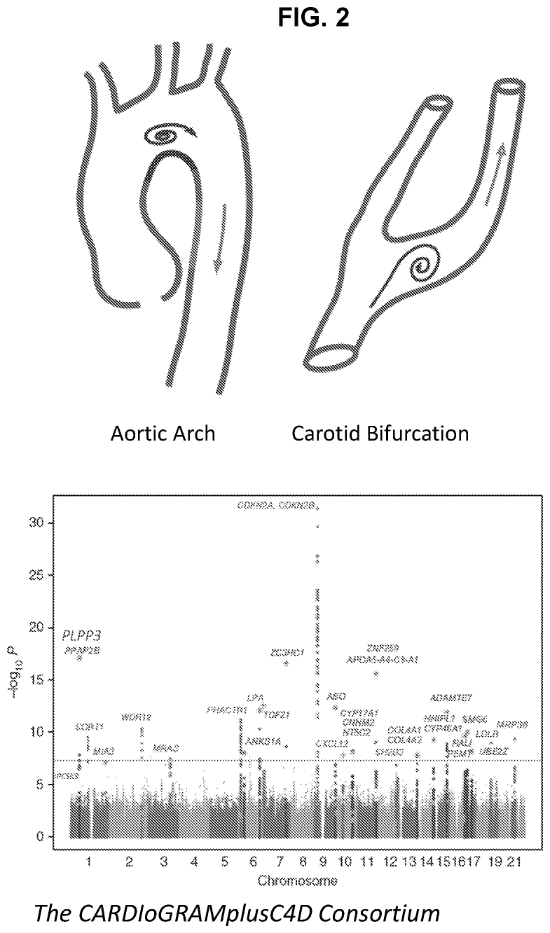

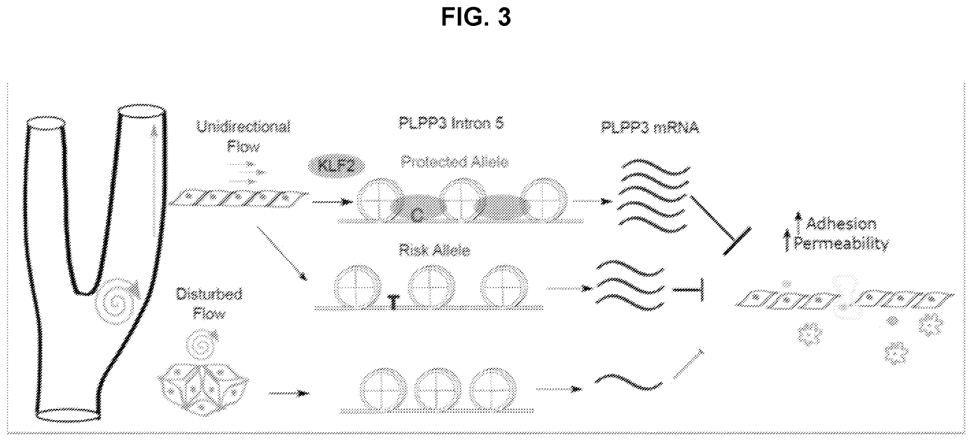

Atherosclerotic disease is the leading cause of morbidity and mortality worldwide. Atherosclerosis, thickening and hardening of the vascular walls, develops preferentially at sites of curvature, branching, and bifurcation in blood vessels where endothelial cells are activated by local disturbed flow. Genome-wide association studies (GWAS) have identified chromosome 1p32.2 (labeled as PLPP3/PPAP2B) as one of the most strongly associated human loci with susceptibility to atherosclerosis (see FIGS. 2 and 3).

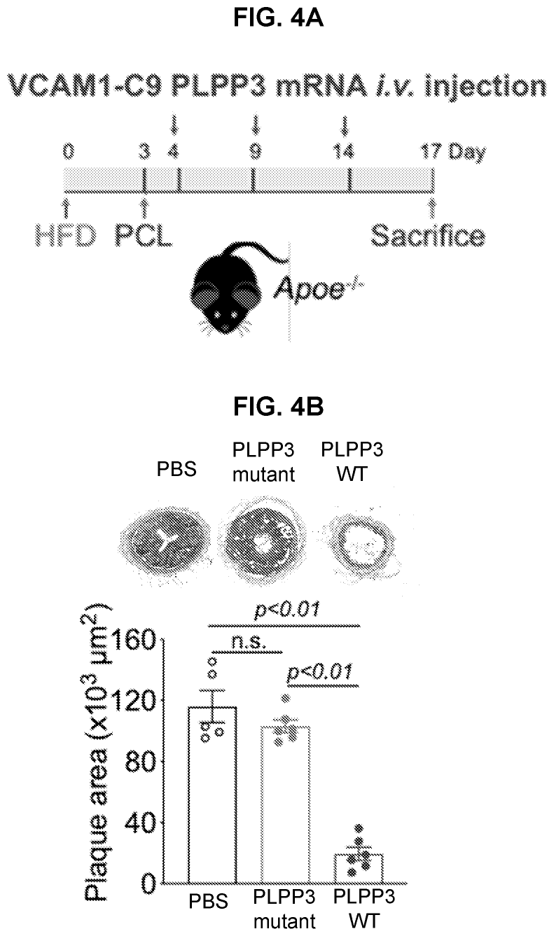

VCAM1-targeting lipid nanoparticles encapsulating functional PLPP3 mRNA significantly reduced disturbed flow-induced atherosclerosis/stenosis in the ligated left carotid artery in mice. Atherosclerosis/stenosis was induced in the left carotid artery by partial carotid ligation (PCL). Experimental design: Mouse carotid atherosclerosis was induced in the left carotid artery by partial carotid ligation (PCL) in ApoE-deficient mice (Apoe−/−) mice fed with high fat diet (HFD). Functional PLPP3 mRNAs or control non-functional mutant PLPP3 mRNAs encapsulated in the VCAMI-targeting lipid nanoparticles were intravenously injected to the Apoe−/− mice subjected to PCL and HFD. Carotid atherosclerosis in the ligated left carotid artery in Apoe−/− mice was not significantly affected by the three injections of Phosphate-buffered saline (PBS) or VCAMI-targeting lipid nanoparticles encapsulating non-functional mutant PLPP3 mRNAs. In sharp contrast, atherosclerosis was significantly reduced in in Apoe−/− mice subjected to injections of VCAM1-targeting lipid nanoparticles encapsulating functional PLPP3 mRNAs. Three injections of PBS or lipid nanoparticles were conducted (FIG. 4A). PBS: Three injections of PBS; PLPP3-MUT: Three injections of VCAM1-targeting lipid nanoparticles encapsulating mutant (Mut) non-functional PLPP3 mRNA. PLPP3-WT: Three injections of VCAM1-targeting lipid nanoparticles encapsulating wild-type (WT) functional PLPP3 mRNA significantly reduced disturbed flow-induced atherosclerosis/stenosis in the ligated left carotid artery in mice (FIG. 4B).

TABLE 1
Formulations of the lipid nanoparticle library.
Components (molar ratios)

| G0-C14 | DOPE | Cholesterol | DSPE-PEG |
|---|---|---|---|
| 2, 5, 10, or 15 | 15, 20, 25, or 30 | 15, 20, 30, or 40 | 1 |

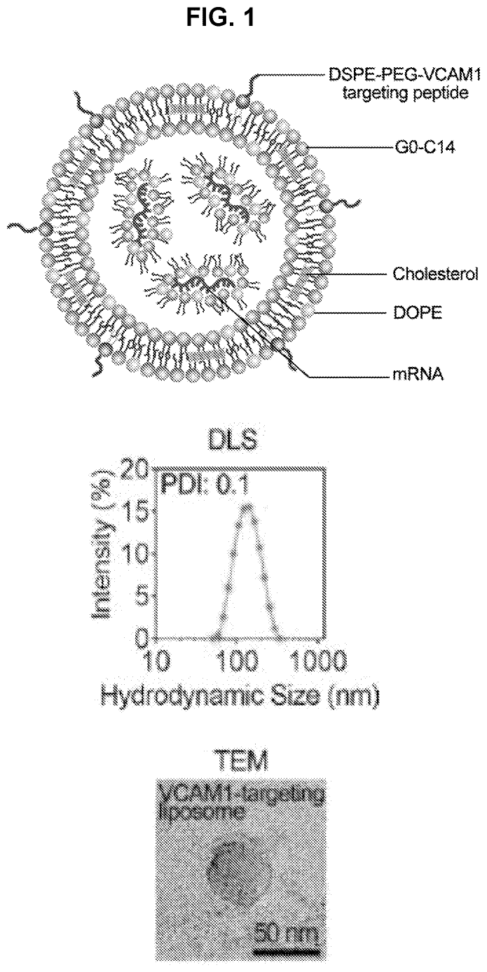

Functionalization and characterization of VCAMI-targeting, PLPP3 mRNA-encapsulated lipid nanoparticles: To understand the functionalization and characterization of VCAM1-targeting, PLPP3 mRNA-encapsulated lipid nanoparticles (see Table 1), the nanoparticle (G0-C14: 5, DOPE: 15; Cholesterol: 25, DSPE-PEG: 1) was prioritized for functionalization to target inflamed vascular endothelium by displaying the VCAM1-binding peptide (VHPKQHR (SEQ ID NO: 1)) on the surface. The VCAM1-targeting peptide was incorporated to the DSPE-PEG molecule (DSPE-PEG-VHPKQHR (SEQ ID NO: 6) in FIG. 1). VHPKQHR (SEQ ID NO: 1) was identified by phage display to specifically bind VCAM-1 in activated endothelium and moreover, was shown to facilitate VCAM1-mediated intracellular internalization of nano-materials in endothelium in vitro and in vivo.

Results demonstrated that VCAM1-targeting lipid nanoparticles encapsulating functional PLPP3 mRNA significantly reduced disturbed flow-induced atherosclerosis/stenosis in the ligated left carotid artery in mice (FIG. 4).

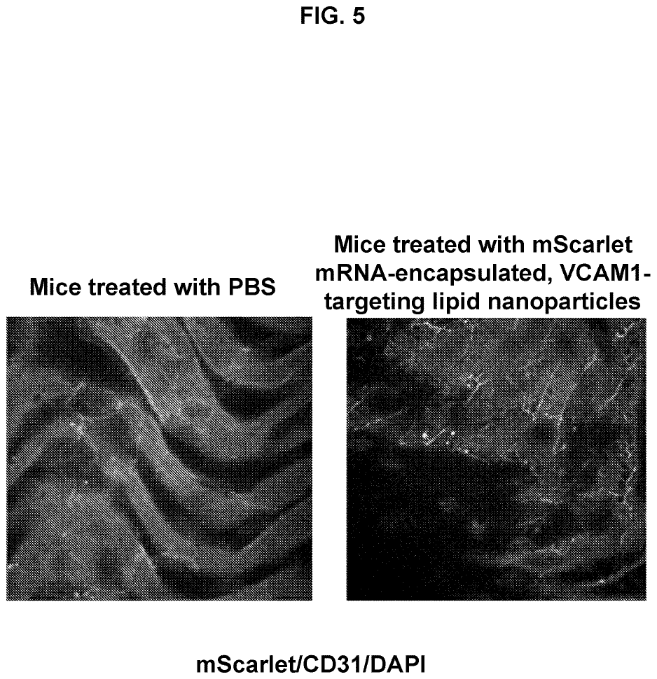

VCAM1-targeting lipid nanoparticle effectively delivered functional mScarlet mRNA to inflamed endothelial cells in diseased arteries in vivo: Partial carotid artery ligation was conducted in mice to induce acute disturbed flow in the left carotid artery (LCA) which leads to endothelial inflammation. En face images of ligated left carotid arteries in mice subjected to an injection of mScarlet mRNA-encapsulated, VCAM1-targeting lipid nanoparticles (FIG. 5). This result demonstrates that the VCAM1-targeting lipid nanoparticles effectively deliver in vivo to inflamed endothelial cells, functional mScarlet mRNA that can be translated to proteins.

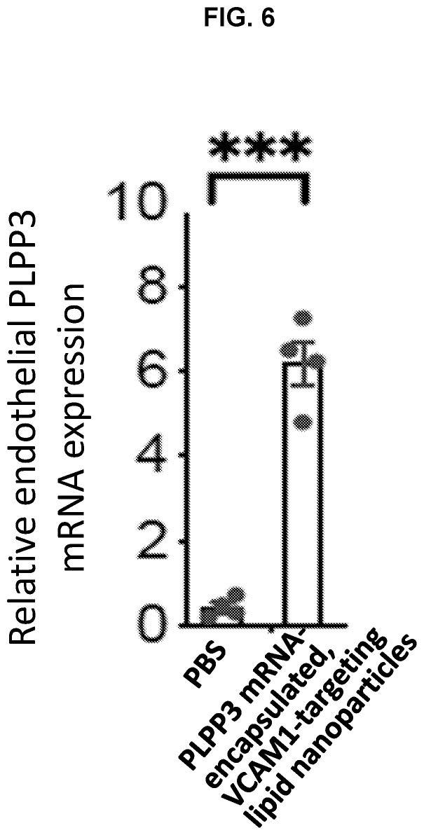

Delivery of functional PLPP3 mRNA to activated vascular endothelium by VCAM1-targeting lipid nanoparticles in vivo: Partial carotid artery ligation was conducted in mice to induce acute disturbed flow in the left carotid artery (LCA) which leads to endothelial inflammation. Real-time PCR detected a significant increase of PLPP3 mRNA expression in inflamed endothelial cells in mice subjected to an injection of PLPP3 mRNA-encapsulated, VCAM1-targeting lipid lipid nanoparticles (FIG. 6). Specifically, PLPP3 mRNA is significantly increased in the inflamed endothelial cells in the ligated carotid artery where local disturbed blood flow activates endothelial cells

### Example 2. MiR-145 Mimic-Encapsulated, PDGFRB-Targeting Polyelectrolyte Complex Micelles Effectively Deliver miR-145 Mimics to Smooth Muscle Cells

Synthesis of PDGFRB targeting PEG-b-pLysine (PDGFRB PEG-pLysine): Polyelectrolyte complex micelles have great potential as gene-delivery vehicles due to the ability to encapsulate negatively charged nucleic acids forming a core by neutralizing the charge, while simultaneously protecting the nucleic acids from nonspecific interactions and enzymatic degradation. The PDGFRB targeting PEG-b-pLysine (5k-50) were synthesized via copper-catalyzed azide-alkyne cycloaddition between alkyne-functionalized peptides targeting platelet derived growth factor receptor beta (alkyne-PDGFRB) and azido-poly (ethylene glycol)-block-poly(L-lysine hydrochloride) (azide-PEG-b-pLysine). For each individual reaction, 2.5 μL of CuSO₄ solution (20 mM in water) was mixed with 5 μL of THPTA solution (50 mM in water). 10 μL of alkyne-PDGFRB (2.5 mM in water) was added to 422.5 μL of potassium phosphate buffer (100 mM, pH 7), 10 μL of azide-PEG-b-pLysine solution (5 mM in water), 7.5 μL of CuSO₄ and THPTA premixed solution, 25 μL of aminoguanidine hydrochloride (100 mM in water), 25 μL of freshly prepared sodium ascorbate (100 mM in water) into a 1.5 mL EP tube, and placed in a sealed EP tube on Vortex mixer at ~3000 rpm to react 18 hours under room temperature. After 18 hours, the solution was dialyzed against DI water with MWCO 2,000 g/mol for 7 days, followed by lyophilization to obtain PDGFRB-PEG-pLysine.

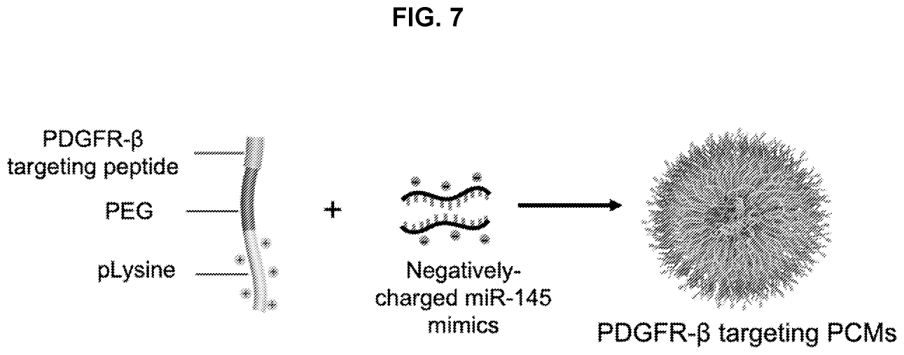

Formation of PDGFRB-targeting polyelectrolyte complex micelles encapsulated with miR-145 mimics (PDGFRB-targeting PCMs) (FIG. 7): 6.89 μL of 100 μM PDGFRB-PEG-pLysine stock solution was mixed with 23.11 μL of nuclease free water, and 20 μL of 20 μM miRNA 145 mimics solution was added and pipetted thoroughly under room temperature, and equilibrated for another 30 minutes.

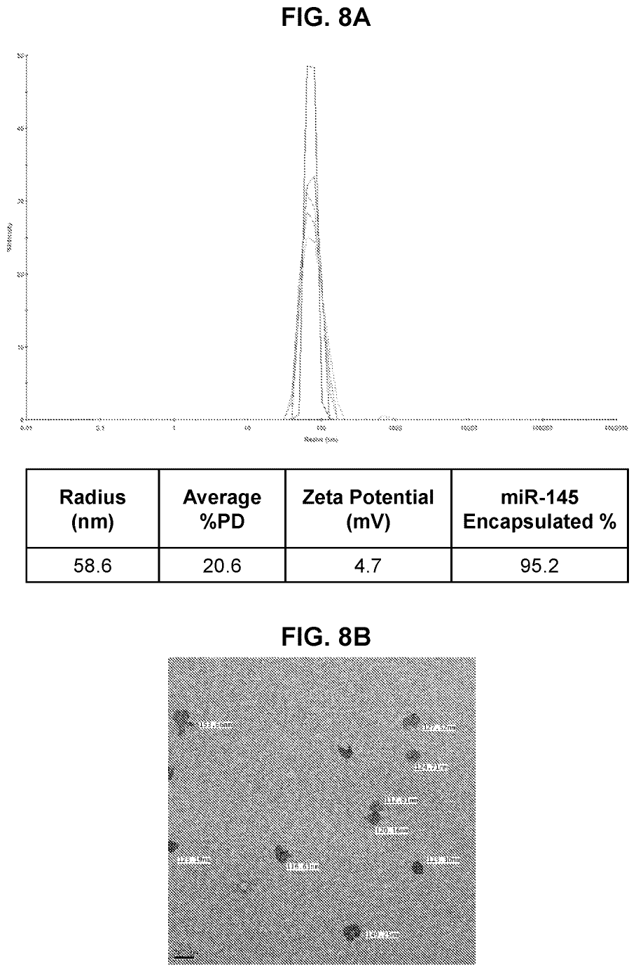

Characterization of the PDGFRB-targeting polyelectrolyte complex micelles (PCMs) that encapsulate small RNAs (FIG. 8): Hydrodynamic radius of miR-145 mimics encapsulated, PDGFRB-targeting PCMs distributed between ~20 to 80 nm as demonstrated by the Dynamic light scattering (DLS) and Transmission electron microscopy (TEM). Dynamic light scattering (DLS) was collected using Wyatt Technology's Mobius™ with 532 nm laser at a constant detector angle of 163.5 degrees under simultaneous DLS and PALS mode with 5s acquisition time at 2V amplitude, 10 Hz for 5 repeated measurements at 25° C. The average radius of PCMs is 58.6±4 nm, with percent and the zeta potential of PCMs is 4.7±2 mV (FIG. 8A). The encapsulation percentage of miR-145 mimics was characterized by RiboGreen free RNA detection assay resulting in a 95.2% encapsulation efficiency. Transmission electron microscopy (TEM) image of PDGFRB-targeting PCMs was collected with Ted Pella carbon coated 400 mesh copper grids using FEI Tecnai F30 electron microscope at 300 kV (FIG. 8B).

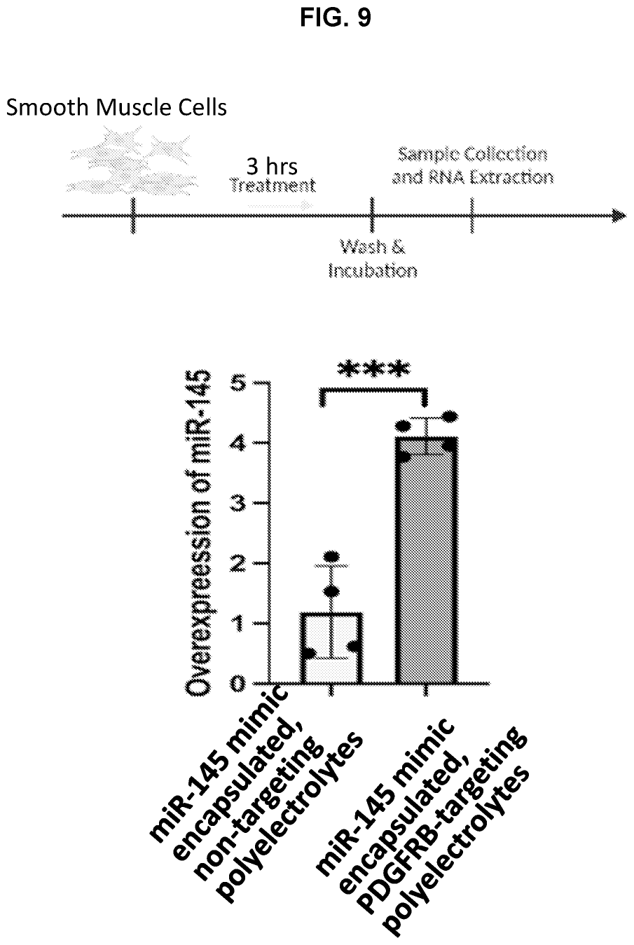

miR-145 mimic encapsulated, PDGFRB-targeting polyelectrolyte complex micelles (PCM) effectively achieved targeted delivery of miR-145 mimics to smooth muscle cells in vitro: Complete media were replaced with serum-free media for human aortic smooth muscle cells (hASMCs) 30 minutes before micelle treatment of 200 nM. hASMCs were treated with PDGFRB-targeting polyelectrolyte complex micelles encapsulating miR-145 mimics. After 3 hours of transfection period, the media were switched back to complete media supplemented with 5% FBS and incubated at 37 C and 5% CO² for another 21 hours. Total RNA was extracted and purified with Qiagen RNeasy Kit, subsequently 100 ng of total mRNA were reverse transcribed to cDNA using Applied Biosystems™ high-capacity cDNA reverse transcription kit together with 5×U6 and miR-145 primer from Taqman™ microRNA assays. Quantitative real-time PCR (qPCR) was performed on LightCycler 480 II (Roche) with 20×U6 and miR-145 probe from Taqman™ microRNA assays and 2× LightCycler 480 probe master. More than three times higher expression of miR-145 was detected in hASMCs with the treatment of miR-145 mimics encapsulated, PDGFRB-targeting PCMs compared to those in hASMCs treated with miR-145 mimics encapsulated in non-targeting PCMs (FIG. 9).

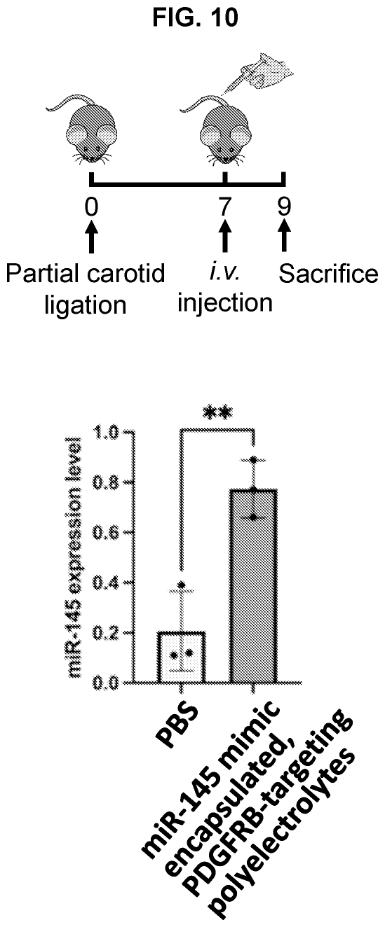

PDGFRB-targeting polyelectrolyte complex micelles (PCM) effectively deliver miR-145 mimics to smooth muscle cell-enriched arterial media and adventitia in vivo in mouse carotid artery subjected to partial carotid ligation. Partial carotid artery ligation was performed on male Apoe−/− mice to induce pathological vascular remodeling in the left carotid artery in seven days. 2 mg/kg of miR-145 mimics encapsulated in PDGFRB-targeting PCMs was injected through tail vein and all mice were sacrificed after 2 days of injection. Total RNA from media and adventitia was extracted and 60 ng of total mRNA were then reverse transcribed to cDNA for quantitative real-time PCR analyses. A significant increase in expression of miR-145 was detected in the media/adventitia of the ligated carotid artery in mice administered with the miR-145 mimics-encapsulated, PDGFRB targeting PCMs compared to those in mice administered with PBS control (FIG. 10).

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

TABLE 2
Sequences.

| Sequence | SEQ ID NO: |
|---|---|
| VHPKQHR | 1 |
| QAHPHVD | 2 |
| DITWDQLWDLMK | 3 |
| CSRNLIDC | 4 |
| CTSDHAVC | 5 |
| (DSPE-PEG)-VHPKQHR | 6 |
| (DSPE-PEG)-DITWDQLWDLMK | 7 |

What is claimed is:

1. A lipid nanoparticle, comprising:
a) a VCAM-1 targeting molecule; and
b) an RNA molecule encoding Phospholipid Phosphatase 3 (PLPP3).

2. The lipid nanoparticle of claim 1, wherein the lipid nanoparticle comprises:
a) a polyamidoamine (PAMAM) dendrimer (G0-C14),
b) cholesterol,
c) polyethylene glycol 2000 (PEG),
d) 1,2-Distearoyl-sn-glycero-3-phosphoethanolamine-Poly(ethylene glycol) (DSPE-PEG), and
e) dioleoylphospha-tidylethanolamine (DOPE), and
wherein the lipid nanoparticle encapsulates the RNA molecule encoding PLPP3.

3. The lipid nanoparticle of claim 2, wherein the DSPE-PEG comprises a PEG domain comprising PEG having an average molecular weight of about 1,000 to about 100,000 Daltons.

4. The lipid nanoparticle of either claim 2 or claim 3, wherein the VCAM-1 targeting molecule comprises a peptide comprising the amino acid sequence VHPKQHR (SEQ ID NO: 1, or DITWDQLWDLMK (SEQ ID NO: 3).

5. The lipid nanoparticle of claim 4, wherein the VCAM-1 targeted lipid nanoparticle comprises DSPE-PEG-VHPKQHR (SEQ ID NO: 6) or DSPE-PEG-DITWDQLWDLMK (SEQ ID NO: 7).

6. The lipid nanoparticle of claim 1, wherein the RNA molecule encoding PLPP3 is mRNA.

7. The lipid nanoparticle of claim 6, wherein the PLPP3 mRNA is an m1Ψ-substituted PLPP3 mRNA.

8. The lipid nanoparticle of any one of claims 1-7, wherein the lipid nanoparticle encapsulates about 0.01 μM to about 100 μM of the RNA molecule encoding PLPP3.

9. The lipid nanoparticle of any one of claims 1-8, wherein the molar ratio of:
a) the polyamidoamine (PAMAM) dendrimer (G0-C14) to DSPE-PEG is about 2:1 to about 15:1;
b) cholesterol to DSPE-PEG is about 15:1 to about 40:1; and
c) DOPE to DSPE-PEG is about 15:1 to about 30:1.

10. A pharmaceutical composition, comprising:
a) a therapeutically effective amount of a lipid nanoparticle comprising a VCAM-1 targeting molecule and an RNA molecule encoding PLPP3; and
b) a pharmaceutically acceptable carrier, solvent, adjuvant, and/or diluent.

11. The pharmaceutical composition of claim 10, wherein the pharmaceutical composition is formulated for inhalation, insufflation, oral, intravenous, topical, ocular, buccal, systemic, nasal, injection, transdermal, rectal, or vaginal administration.

12. The pharmaceutical composition of either claim 10 or claim 11, wherein the pharmaceutical composition is formulated for inhalation or intramuscular, intraperitoneal, subcutaneous, or intravenous administration.

13. A pharmaceutical composition for cardiovascular delivery of RNA comprising:
a) a lipid nanoparticle comprising
i) a VCAM-1 targeting molecule; and
ii) an RNA molecule encoding PLPP3; and
b) a pharmaceutically acceptable carrier,
wherein the composition is formulated such that once administered to the cardiovascular system, it results in the delivery of the RNA molecule to a subject's cardiovascular system.

14. A method of treating an arterial disease in a subject, comprising:
a) administering to the subject a therapeutically effective amount of a pharmaceutical composition comprising a lipid nanoparticle comprising a VCAM-1 targeting molecule and an RNA molecule encoding PLPP3, wherein the lipid nanoparticle is preferentially targeted to inflamed vascular endothelial cells associated with the arterial vascular disease; and
b) reducing inflammation at the site of the inflamed vascular endothelial cells.

15. The method of claim 14, wherein the arterial disease comprises one or more of atherosclerosis, peripheral artery disease, ischemic stroke, and stenosis.

16. The method of either claim 14 or claim 15, wherein the method results in one or more of an increase of at least about 2 fold to at least about 1000 fold of the RNA encoding PLPP3 compared to a control and/or an increase of at least about 2 fold to at least about 200 fold of PLPP3 protein compared to a control at the site of the inflamed endothelial cells.

17. The method of any one of claims 14-16, wherein the probability of survival of the subject is at least about 10% greater than an expected probability of survival without administration of the pharmaceutical composition.

18. A polyelectrolyte complex micelle (PCM), comprising:
a) a PDGFRB targeting molecule; and
b) an RNA molecule.

19. The polyelectrolyte complex micelle of claim 18, wherein the polyelectrolyte complex micelle comprises:
a) a polyethylene glycol (PEG) domain, and
b) poly-lysine,
wherein the RNA molecule is a microRNA.

20. The polyelectrolyte complex micelle of claim 18, wherein the PEG domain comprises PEG having an average molecular weight of about 1,000 to about 100,000 Daltons.

21. The polyelectrolyte complex micelle of any one of claims 18-20, wherein the PDGFRB targeting molecule comprises a peptide comprising the amino acid sequence CSRNLIDC (SEQ ID NO: 4).

22. The polyelectrolyte complex micelle of any one of claims 18-21, wherein the polyelectrolyte complex micelle comprises CSRNLIDC (SEQ ID NO: 4), the PEG domain, and poly-lysine, wherein the polyelectrolyte complex micelle encapsulates the RNA molecule.

23. The polyelectrolyte complex micelle of any one of claims 18-22, wherein the RNA molecule is a microRNA encoding a microRNA145 mimic.

24. The polyelectrolyte complex micelle of any one of claims 18-23, wherein the polyelectrolyte complex micelle encapsulates about 0.01 μM to about 100 μM of the RNA molecule.

25. A pharmaceutical composition, comprising:
a) a therapeutically effective amount of a polyelectrolyte complex micelle comprising a PDGFRB targeting molecule, a PEG domain, and poly-lysine, wherein the polyelectrolyte complex micelle encapsulates the RNA molecule; and
b) a pharmaceutically acceptable carrier, solvent, adjuvant, and/or diluent.

26. The pharmaceutical composition of claim 25, wherein the PEG domain comprises PEG having an average molecular weight of about 1,000 to about 100,000 Daltons.

27. The pharmaceutical composition of either claim 25 or claim 26, wherein the PDGFRB targeting molecule comprises a peptide comprising the amino acid sequence CSRNLIDC (SEQ ID NO: 4).

28. The pharmaceutical composition of any one of claims 25-27, wherein the RNA molecule is a microRNA encoding a microRNA145 mimic.

29. The pharmaceutical composition of any one of claims 25-28, wherein the polyelectrolyte complex micelle encapsulates about 0.01 μM to about 100 μM of the RNA molecule.

30. The pharmaceutical composition of any one of claims 25-29, wherein the pharmaceutical composition is formulated for inhalation, insufflation, oral, intravenous, topical, ocular, buccal, systemic, nasal, injection, transdermal, rectal, or vaginal administration.

31. The pharmaceutical composition of any one of claims 25-29, wherein the pharmaceutical composition is formulated for inhalation or intramuscular, intraperitoneal, subcutaneous, or intravenous administration.

32. A pharmaceutical composition for cardiovascular delivery of an RNA molecule comprising:
a) a polyelectrolyte complex micelle comprising:
i) a PDGFRB targeting molecule comprising the amino acid sequence CSRNLIDC (SEQ ID NO: 4);
ii) a PEG domain; and
iii) poly-lysine;
wherein the RNA molecule is a microRNA encoding a microRNA145 mimic
b) a pharmaceutically acceptable carrier,
wherein the composition is formulated such that once administered to the cardiovascular system, it results in the delivery of the RNA molecule to a subject's cardiovascular system.

33. A method of treating an arterial disease in a subject, comprising:
a) administering to the subject a therapeutically effective amount of the pharmaceutical composition of any one of claims 25-32, wherein the polyelectrolyte complex micelle is preferentially targeted to smooth muscle cells (SMC) associated with the arterial vascular disease; and
b) reducing SMC proliferation and migration, decreasing synthetic SMC phenotype, and increasing the contractile phenotype of SMC at the site of diseased blood vessels.

34. The method of claim 33, wherein the arterial disease comprises one or more of atherosclerosis, peripheral artery disease, ischemic stroke, and stenosis.

35. The method of either claim 33 or claim 34, wherein the method results in one or more of an increase of at least about 2 fold to at least about 1000 fold of the miRNA145 in smooth muscle cells compared to a control at the site of vascular disease.

36. The method of any one of claims 33-35, wherein the probability of survival of the subject is at least about 10% greater than an expected probability of survival without administration of the pharmaceutical composition.


SEQUENCE LISTING (7 sequences)

SEQ ID NO: 1 - protein, 7 aa, synthetic construct
VHPKQHR

SEQ ID NO: 2 - protein, 7 aa, synthetic construct
QAHPHVD

SEQ ID NO: 3 - protein, 12 aa, synthetic construct
DITWDQLWDLMK

SEQ ID NO: 4 - protein, 8 aa, synthetic construct
CSRNLIDC

SEQ ID NO: 5 - protein, 8 aa, synthetic construct
CTSDHAVC

SEQ ID NO: 6 - protein, 7 aa, synthetic construct
  MOD_RES 1: note = DSPE-PEG on the N-terminal of the first residue
VHPKQHR

SEQ ID NO: 7 - protein, 12 aa, synthetic construct
  MOD_RES 1: note = DSPE-PEG on the N-terminal of the first residue
DITWDQLWDLMK